(19) United States
(12) Patent Application Publication
(10) Pub. No.: US 2011/0282225 A1
(43) Pub. Date: Nov. 17, 2011

(54) TECHNIQUES FOR REVIEWING AND ANALYZING IMPLANTABLE MEDICAL DEVICE SYSTEM DATA

(75) Inventors: Katherine H. Anderson, Golden Valley, MN (US); Timothy P. LaCroix, Buffalo, MN (US); Manfred Justen, Lino Lakes, MN (US); John W. Komp, Brooklyn Park, MN (US); Arch W. Butler, Minneapolis, MN (US); Christopher M. Petersen, Ham Lake, MN (US); Laura K. Dove, Minneapolis, MN (US); David L. Holmstrom, Fridley, MN (US); James D. Webb, Maple Grove, MN (US)

(73) Assignee: Medtronic, Inc., Minneapolis, MN (US)

(21) Appl. No.: 12/778,842

(22) Filed: May 12, 2010

(51) Int. Cl. A61B 5/0402

(52) U.S. Cl. 600/510

(57) ABSTRACT

A computing device includes a memory device, a communication module, an interface, a processor, a display controller, and an input device. The communication module wirelessly receives a stream of first electrogram waveforms (EGMs) from an implantable medical device (IMD). The interface receives a stream of second EGMs. The processor stores the first and second EGMs and retrieves the stored first and second EGMs. The display controller displays the first and second EGMs together when the first and second EGMs are received. The input device is configured to receive a selection command from a user. The processor retrieves a portion of at least one of the first and second stored EGMs in response to the selection command. The display controller displays the retrieved portion of the at least one of the first and second stored EGMs while the processor concurrently stores the streams of the first and second EGMs.

### TECHNICAL FIELD

The disclosure relates to systems and methods for evaluating data retrieved from an implantable medical device system, and, more particularly, to systems and methods for storing, viewing, and analyzing data retrieved from an implantable medical device system.

### BACKGROUND

Some implantable medical devices (IMDs), such as cardiac pacemakers or cardioverter-defibrillators, provide therapeutic electrical stimulation to a patient via electrodes carried by one or more implantable leads. Some implantable medical devices additionally or alternatively sense physiological parameters of the patient, e.g., via sensors on or within the device, or carried by one or more implantable leads coupled to the device, or by sensing electrical signals via electrodes on or within the device, or carried by one or more implantable leads coupled to the device. The functioning of IMDs and the state of the patient may both be regularly assessed during follow-up sessions subsequent to implantation. Functions of the IMD tested during a follow-up session may include therapy delivery capabilities, sensing capabilities, and lead impedance associated with the IMD.

During a follow-up session for a cardiac pacemaker or cardioverter-defibrillator, for example, a clinician may review cardiac electrogram (CEGM) waveforms and other data received from the IMD and electrocardiography (ECG) waveforms received from an ECG device. The CEGM/ECG waveforms may be printed on strip charts during the follow-up. Accordingly, the follow-up procedure may be documented for subsequent analysis on one or more paper strip charts.

The strip chart printers used during the follow-up may continuously print the CEGM/ECG waveforms during the procedure. Since not all of the CEGM/ECG waveforms printed on the strip charts may be useful for analysis, continuous printing of the waveforms during a procedure may produce substantial amounts of information that may not be of interest. Searching through the substantial amounts of information produced may impede efficient analysis of the actual areas of interest. Furthermore, the amount of paper generated may not be cost efficient.

Some IMD programmers may not continuously print the CEGM/ECG waveforms, but may allow for selective printing or storing of the CEGM/ECG waveforms. Selective printing or storing of CEGM/ECG waveforms may be accomplished, for example, using a “freeze” command, or other selective printing or storing command. The freeze command may freeze the waveforms on the display of the programmer, allowing the user to analyze the CEGM/ECG waveforms and print and store the waveforms if desired. However, when using the freeze command, the user may be required to operate the programmer while simultaneously interacting with the patient. Such multitasking undertaken by the user may reduce the user's ability to detect and freeze areas of interest in the CEGM/ECG waveforms. Accordingly, areas of interest may pass by the user without being frozen. Additionally, some areas of interest may not be saved when using the freeze command because the areas of interest may occur while the CEGM/ECG waveforms are frozen. In either case, the areas of interest that are lost may not be recreated easily, and accordingly, the areas of interest may not be retrievable for subsequent analysis if not frozen at the time of occurrence using the freeze command.

### SUMMARY

In general, the disclosure is directed to a computing device for storing (e.g., continuously storing) and/or displaying waveforms, such as electrogram waveforms, and/or other data received from an implantable medical device (IMD). The waveforms and/or other data received from the IMD may be received in conjunction with and correlated with waveforms, such as electrogram waveforms, and/or other data received from another device (e.g., an external device). Accordingly, the computing device may facilitate user review and analysis of the waveforms or other data from the IMD and the other device during follow-up evaluation of the IMD. In one example, the computing device stores, correlates, and displays cardiac electrogram (CEGM) and electrocardiography (ECG) waveforms, and may also display other data from the IMD, such as marker channel data.

In one example, the computing device receives a stream of CEGM waveforms from the IMD and a stream of ECG waveforms from an external ECG device. The computing device stores the CEGM and ECG waveforms and displays the CEGM and ECG waveforms on a display. The computing device also includes an input device that a user may interact with in order to view stored CEGM waveforms on the display while the programmer concurrently stores the stream of CEGM waveforms.

Thus, the computing device may display received waveforms and other data in real-time, and may also display stored waveforms while concurrently storing the stream of received waveforms. Accordingly, a clinician may review waveforms on the display while still recording the stream of waveforms received from the IMD and external device. The clinician may also review the entire recording session at a later time by retrieving the stored waveforms from a memory. Therefore, the clinician may be able to simultaneously interact with the patient while operating the computing device without missing any areas of interest present in the waveforms. Subsequent to review on the display of the computing device, the clinician may print out the areas of interest. Accordingly, the computing device may eliminate the substantial amount of paper that may accompany a follow-up while also eliminating the chance of missing areas of interest in the waveforms.

Additionally, using the input device, the user may annotate the real-time and/or stored waveforms using an annotation function. For example, the computing device may receive freehand and/or alphanumeric annotations using at least one of a mouse, a keyboard, a stylus, or a touchscreen. Accordingly, the user may retain the benefits of using paper, such as freehand annotations, while also minimizing the inconveniences associated with producing substantial amounts of paper records.

The computing device of the present disclosure may also include additional functions for analyzing and reviewing data received from the IMD and the external device. For example, the additional functions may include, but are not limited, a measurement function, an attachment annotation function, a searching function, a waveform comparison function, a timing diagram function, and a medical record generation function. The user may access the additional functions of the computing device using a user interface of the computing device.

The measurement function allows the user to measure the waveforms on the display. For example, the measurement function may allow for measurement of the real-time waveforms and/or the stored waveforms on the display. The attachment annotation function may allow for files of various formats to be stored and associated with a waveform. The searching function may allow a user to search waveforms based on a title associated with the waveform. The comparison function may allow the user to compare two separate waveforms on the display. Using the comparison function, the user may compare any combination of stored waveforms and real-time waveforms. The medical record generation function may format the waveforms and other data, e.g., CEGM waveforms, the ECG waveforms, and the marker channel data, into a format that may be stored in an electronic medical record database. The computing device may upload the formatted data to an electronic medical record database.

In one example according to the present disclosure, a computing device comprises a memory device, a communication module, an interface, a processor, a display controller, and an input device. The communication module wirelessly receives a stream of first electrogram (EGM) waveforms of a patient from an IMD. The interface receives a stream of second EGM waveforms of the patient that are based on external measurement of changes in electrical potential associated with the patient. The processor stores the first and second EGM waveforms in the memory device and retrieves the stored first and second EGM waveforms from the memory device. The display controller displays the first and second EGM waveforms together on a display when the first and second EGM waveforms are received. The input device is configured to receive a selection command from a user. The processor retrieves a portion of at least one of the first and second stored EGM waveforms from the memory device in response to the selection command. The display controller displays the retrieved portion of the at least one of the first and second stored EGM waveforms on the display while the processor concurrently stores the streams of the first and second EGM waveforms in the memory device. The first EGM waveforms may comprise CEGM waveforms from the IMD and the second EGM waveforms may comprise ECG waveforms.

In another example according to the present disclosure, the communication module is configured to wirelessly receive marker channel data and other data from the IMD that indicates the occurrence and timing of therapy and sensing events associated with the IMD. The processor is configured to store the marker channel data and other data in the memory device and retrieve the stored marker channel data and other data from the memory device. The display controller is configured to display the marker channel data and other data on the display together with the first and second EGM waveforms when the marker channel data and other data are received. The processor retrieves a portion of the marker channel data and other data from the memory device in response to the selection command. The display controller displays the retrieved portion of the marker channel data and other data on the display while the processor concurrently stores received marker channel data and other data in the memory device.

In another example according to the present disclosure, the input device is configured to receive an annotation from a user. The display controller displays the annotation on a selected portion of one of the CEGM and ECG waveforms displayed while the processor concurrently stores the streams of CEGM and ECG waveforms in the memory device. The annotation may be a freehand annotation such as a freehand drawing when the input device includes at least one of a mouse, a stylus, and a touchscreen. The annotation may be an alphanumeric annotation when the input device includes an alphanumeric keypad.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a conceptual diagram of an example diagnostic system comprising an implantable medical device (IMD) for delivering stimulation therapy to a heart of a patient via implantable leads.

FIG. 2 is a conceptual diagram the IMD and the implantable leads of the system of FIG. 1 in conjunction with the heart.

FIG. 3 is a conceptual diagram of the IMD of FIG. 1 coupled to a different configuration of leads.

FIG. 4 is a functional block diagram illustrating an example configuration of the IMD of FIG. 1.

FIG. 5 is a functional block diagram of an example programmer according to the present disclosure.

FIG. 6 is an example graphic-user interface (GUI) implemented on the example programmer of FIG. 5 according to the present disclosure.

FIG. 7 is another example format of a GUI implemented on the example programmer of FIG. 5 according to the present disclosure.

FIG. 8 illustrates an example searching function of the GUI of FIG. 7 according to the present disclosure.

FIG. 9 illustrates an example ladder diagram that may be generated and displayed according to the present disclosure.

FIG. 10 is a flow diagram illustrating an example method for reviewing and analyzing electrogram data according to the present disclosure.

### DETAILED DESCRIPTION

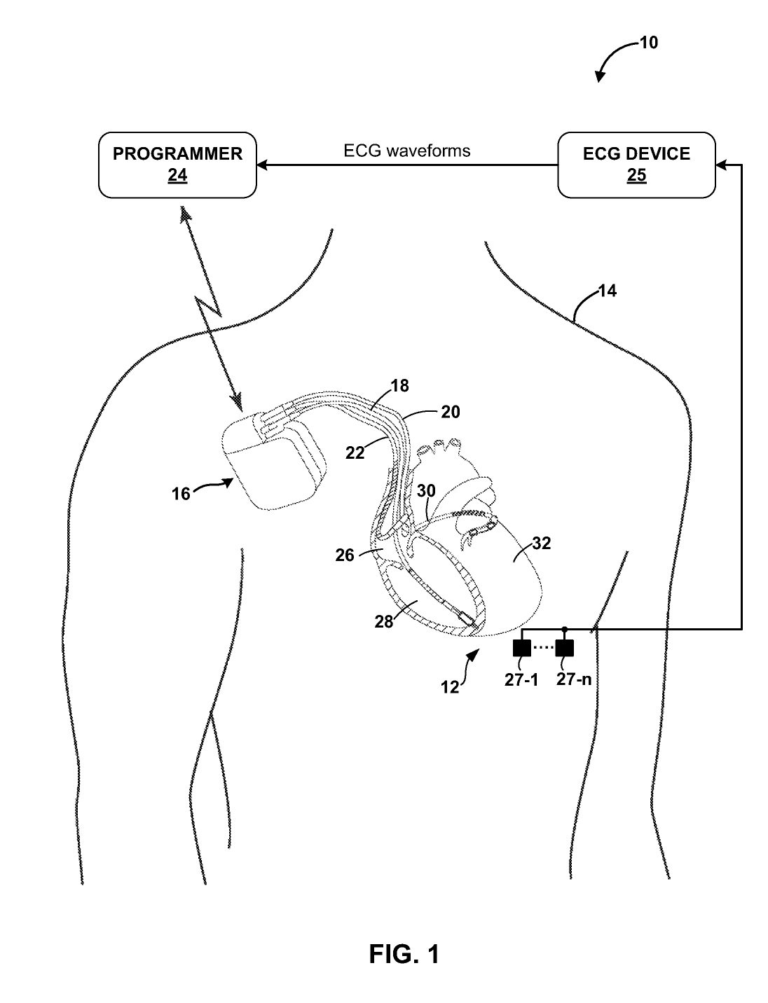

FIG. 1 is a conceptual diagram of an example diagnostic system 10 that may be used to provide therapy to a heart 12 of a patient 14. The diagnostic system 10 includes an IMD 16, which is coupled to leads 18, 20, and 22. For example, the IMD 16 may be an implantable pacemaker, cardioverter, and/or defibrillator that provides electrical signals to the heart 12 using one or more of the leads 18, 20, 22.

The leads 18, 20, 22 extend into the heart 12 of the patient 14. The leads 18, 20, 22 sense electrical activity of the heart 12 and/or deliver electrical stimulation to the heart 12. Right ventricular (RV) lead 18 extends into the right ventricle 28 of the heart 12 through one or more veins (not shown), the superior vena cava (not shown), and the right atrium 26. Left ventricular (LV) coronary sinus lead 20 extends through one or more veins, the vena cava, the right atrium 26, and into the coronary sinus 30 to a region adjacent to the free wall of left ventricle 32 of the heart 12. Right atrial (RA) lead 22 extends into the right atrium 26 of the heart 12 through one or more veins and the vena cava.

The diagnostic system 10 includes a programmer 24 that communicates with the IMD 16. The programmer 24 may be a handheld computing device, desktop computing device, a networked computing device, etc. Accordingly, the programmer 24 may be a computing device that includes a computer-readable storage medium having instructions that cause a processor of the programmer 24 to provide the functions attributed to the programmer 24 in the present disclosure.

The programmer 24 may retrieve data stored in the IMD 16 and/or program the IMD 16 to provide various therapies. Accordingly, a user may retrieve data from the IMD 16 and program the IMD 16 using the programmer 24. For example, the user may include a physician, a technician, a surgeon, an electrophysiologist, or other clinician. Data retrieved from the IMD 16 using the programmer 24 includes waveforms that indicate electrical activity of the heart 12. The waveforms retrieved from the IMD 16 may be referred to as cardiac electrogram (CEGM) waveforms or, more particularly in cases in which the waveforms are detected via an intracardiac electrode, intracardiac electrogram (IEGM) waveforms. The programmer 24 may continuously receive CEGM waveforms from the IMD 16 when communicating with the IMD 16. In other words, the programmer 24 may receive a stream of CEGM waveforms from the IMD 16.

In some implementations, the diagnostic system 10 may include an electrocardiography device (ECG device) 25 that measures electrical activity of the heart 12 using a plurality of external electrodes, represented as electrodes 27-1 to 27-n. Although only 2 electrodes 27-1 and 27-n are illustrated, the ECG device 25 may be coupled to additional electrodes that are not illustrated. For example, the ECG device 25 may be coupled to up to 12 electrodes in typical applications. The programmer 24 receives samples of the measured electrical activity of the heart 12 (hereinafter “ECG waveforms”) from the ECG device 25. The programmer 24 may continuously receive ECG waveforms from the ECG device 25 when communicating with the ECG device 25. In other words, the programmer 24 may receive a stream of ECG waveforms from the ECG device 25.

Although the programmer 24 is illustrated as communicating with the IMD 16 and the ECG device 25, the programmer 24 may communicate with additional or alternative implantable or external devices that provide electrical stimulation to the patient 14 and/or measure electrical potentials or other physiological signals associated with the patient 14. For example, the programmer 24 may communicate with other forms of cardiac monitors, such as insertable cardiac monitors that monitor heart rhythms and record ECGs, which may be associated with physiological events, e.g., fainting. As a further example, the programmer 24 may communicate with an implantable neurostimulator (not shown) that provides at least one of deep brain stimulation, vagus nerve stimulation, and spinal cord stimulation, for example. In such examples, the programmer 24 may receive an electroencephalogram (EEG) or other neurological electrogram from the implantable neurostimulator, as well as from an external device. In other examples, programmer 24 may receive other signals from other implantable and external medical devices. Accordingly, the programmer 24 may receive a plurality of different electrogram waveforms depending on the devices connected to the programmer 24.

The programmer 24 may receive data from the IMD 16 and the ECG device 25 in real-time. In other words, the programmer 24 may receive data from the IMD 16 as the IMD 16 senses electrical potentials within the heart 12. Real-time data may include, for example, cardiac signals measured by the IMD 16 while the programmer 24 is in communication with the IMD 16. Real-time data may additionally or alternatively include marker channel data, a current rhythm of the heart, or other sensed physiological parameters of the patient 14, such as intracardiac or intravascular pressure, activity, posture, respiration, or thoracic impedance.

The programmer 24 may also receive data from the IMD 16 that is stored in a memory of the IMD 16. For example, stored data may include arrhythmic episode data and trends in the rhythm of the heart 12 over time. As another example, stored data may include other sensed physiological parameters of the patient 14, such as intracardiac or intravascular pressure, activity, posture, respiration, or thoracic impedance. Additionally, stored data may include information regarding the performance or integrity of the IMD 16 or other components of the diagnostic system 10, such as the leads 18, 20, 22, or a power source of the IMD 16.

A user may program the IMD 16 using the programmer 24. Programming the IMD 16 may include, for example, setting values for operational parameters, programming a therapy progression, selecting electrodes used to deliver defibrillation pulses, selecting waveforms for the defibrillation pulses, or selecting or configuring a fibrillation detection algorithm for the IMD 16. The user may also use programmer 24 to program other therapies provided by the IMD 16, such as cardioversion or pacing therapies.

The IMD 16 and the programmer 24 may communicate via wireless communication using any techniques known in the art. Examples of communication techniques may include, for example, low frequency or radiofrequency (RF) telemetry, but other techniques are also contemplated. In some examples, the programmer 24 may include a programming head that may be placed proximate to the patient's body near the IMD 16 implant site in order to improve the quality or security of communication between the IMD 16 and the programmer 24.

The programmer 24 and the ECG device 25 may coupled by a wired or wireless connection. Furthermore, in some examples, the programmer 24 may incorporate, or otherwise provide functionality described herein with respect to an external device that that measures electrical signals within a patient using external electrodes, such as the ECG device 25. In such examples, the programmer 24 may be coupled to a patient via one or more electrodes 27.

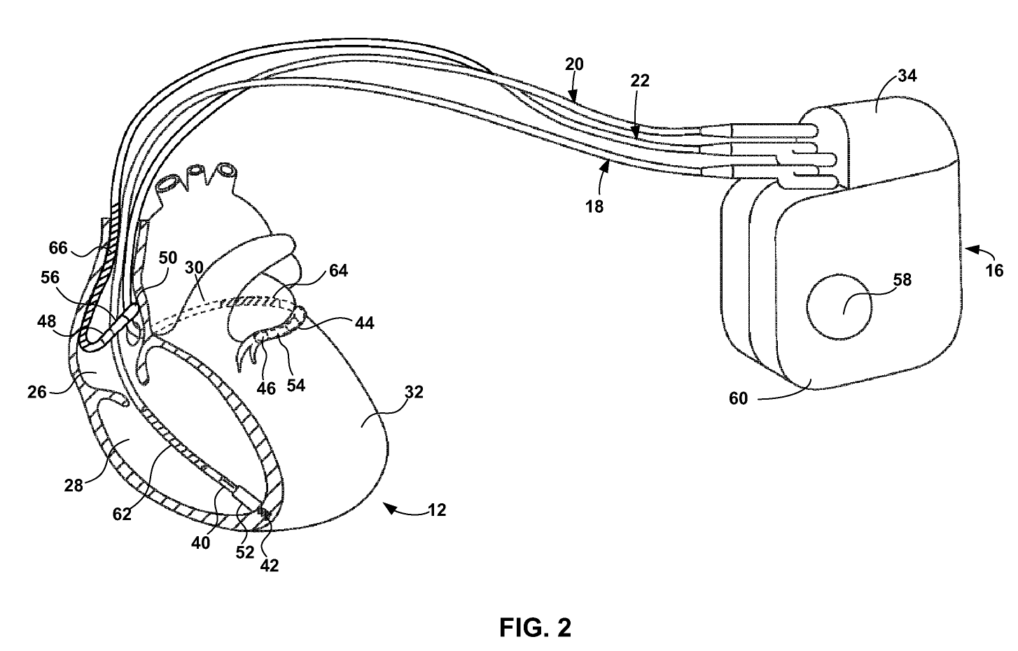

FIG. 2 is a conceptual diagram illustrating the IMD 16 and the leads 18, 20, 22 of the diagnostic system 10 in greater detail. The IMD 16 includes a housing 60 and a connector block 34. The leads 18, 20, 22 are mechanically and electrically coupled to the IMD 16 via the connector block 34. The housing 60 may enclose a signal generator that generates therapeutic stimulation, such as cardiac pacing pulses and defibrillation shocks, as well as a sensing module for monitoring the rhythm of heart 12. The leads 18, 20, 22 are coupled to a signal generator and a sensing module of the IMD 16 via the connector block 34. The IMD 16 may sense electrical signals attendant to the depolarization and repolarization of heart 12 via the leads 18, 20, 22. The IMD 16 may also provide electrical stimulation to the heart 12 via the leads 18, 20, 22.

The IMD 16 may provide pacing pulses to the heart 12 based on the electrical signals sensed within the heart 12. The IMD 16 may also provide defibrillation and/or cardioversion therapy to the heart 12. For example, the IMD 16 may detect arrhythmia of the heart 12, such as fibrillation of the ventricles 28 and 32, and deliver cardioversion or defibrillation therapy to heart 12 in the form of electrical pulses. In some implementations, the IMD 16 may be programmed to deliver a progression of therapies, e.g., pulses with increasing energy levels, until a tachyarrhythmia of the heart 12 is stopped. The IMD 16 may detect tachycardia or fibrillation employing one or more tachycardia or fibrillation detection techniques known in the art.

Leads 18, 20, 22 include electrodes 40, 42, 44, 46, 48, 50, 62, 64, and 66, respectively. The IMD 16 may sense electrical signals via the electrodes 40, 42, 44, 46, 48, 50, 62, 64, and 66. The IMD 16 may also provide electrical stimulation to the heart 12 using the electrodes 40, 42, 44, 46, 48, 50, 62, 64, and 66. Although each of the leads 18, 20, 22 of FIG. 2 includes three electrodes, other configurations of electrodes are contemplated. For example, each of the three leads 18, 20, 22 may include more or less than three electrodes.

Bipolar electrodes 40 and 42 are located adjacent to the distal end of lead 18 in the right ventricle 28. Bipolar electrodes 44 and 46 are located adjacent to the distal end of lead 20 in the coronary sinus 30. Bipolar electrodes 48 and 50 are located adjacent to the distal end of lead 22 in the right atrium 26. There are no electrodes located in the left atrium in the illustrated example, however, other examples may include electrodes in left atrium. Leads 18, 20, 22 also include elongated electrodes 62, 64, 66, respectively, which may take the form of a coil.

The IMD 16 includes a housing electrode 58, which may be formed integrally with an outer surface of the hermetically-sealed housing 60 of the IMD 16 or otherwise coupled to the housing 60. Although a single housing electrode 58 is illustrated in FIG. 2, the IMD 16 may include more or less than a single housing electrode 58.

The IMD 16 may sense electrical signals attendant to the depolarization and repolarization of heart 12 via the electrodes 40, 42, 44, 46, 48, 50, 58, 62, 64, and 66. The IMD 16 may sense such electrical signals via any bipolar combination of electrodes 40, 42, 44, 46, 48, 50, 58, 62, 64, and 66. Furthermore, any of the electrodes 40, 42, 44, 46, 48, 50, 62, 64, and 66 may be used for unipolar sensing in combination with the housing electrode 58.

The IMD 16 may deliver pacing pulses via a unipolar or bipolar combination of electrodes. The IMD 16 delivers pacing pulses via bipolar combinations of electrodes 40, 42, 44, 46, 48 and 50 to produce depolarization of cardiac tissue of the heart 12. The IMD 16 may deliver pacing pulses via any of electrodes 40, 42, 44, 46, 48 and 50 in combination with housing electrode 58 in a unipolar configuration.

The IMD 16 may deliver defibrillation pulses to the heart 12 via any combination of elongated electrodes 62, 64, 66, and housing electrode 58. Electrodes 58, 62, 64, 66 may also be used to deliver cardioversion pulses to the heart 12.

The electrode configuration of the diagnostic system 10 illustrated in FIGS. 1 and 2 is merely one example electrode configuration. In other examples, a diagnostic system may include epicardial leads and/or patch electrodes instead of or in addition to the transvenous leads 18, 20, 22 illustrated in FIG. 1.

Although the IMD 16 of FIG. 1 is coupled to three leads 18, 20, 22, other lead configurations are contemplated. In other words, the number of leads coupled to the IMD 16 and the locations of the leads relative to the heart 12 may vary. For example, in some alternative implementations, the diagnostic system 10 may include an additional lead or lead segment (not shown in FIG. 1) that deploys one or more electrodes within the left atrium, vena cava, or other vein. The additional lead may allow for alternative electrical sensing configurations that may provide improved sensing accuracy in some patients.

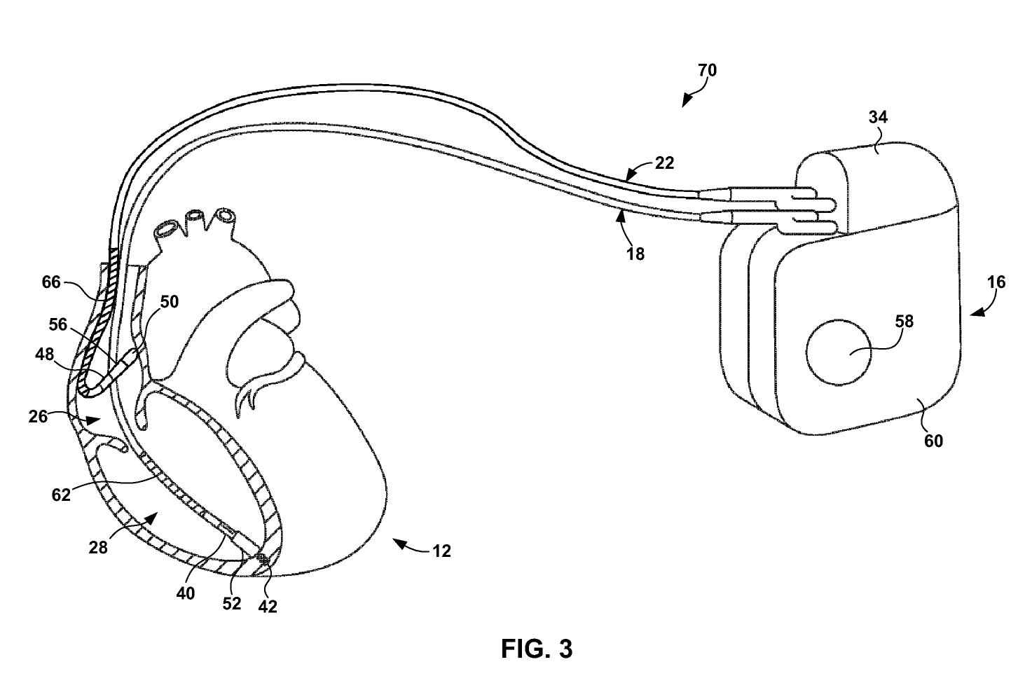

FIG. 3 is a conceptual diagram illustrating another lead configuration. A diagnostic system 70, similar to the diagnostic system 10, includes two leads 18, 22, rather than three leads. The leads 18, 22 are implanted within the right ventricle 28 and the right atrium 26, respectively. The diagnostic system 70 shown in FIG. 3 may be useful for providing defibrillation and pacing pulses to the heart 12. The systems and methods of the present disclosure may also be implemented in the diagnostic system 70.

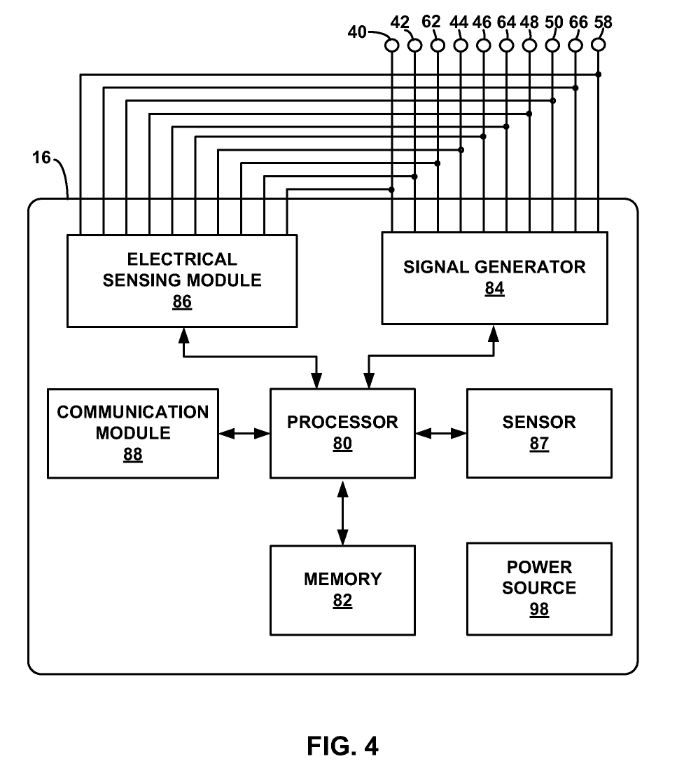

FIG. 4 is a functional block diagram illustrating an example configuration of the IMD 16. The IMD 16 includes a processor 80, memory 82, a signal generator 84, an electrical sensing module 86, a sensor 87, a communication module 88, and a power source 98. Memory 82 may include computer-readable instructions that, when executed by the processor 80, cause the IMD 16 and the processor 80 to perform various functions attributed to the IMD 16 and the processor 80 herein. Memory 82 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other digital media.

The processor 80 may include any one or more of a microprocessor, a microcontroller, a digital signal processor (DSP), an application specific integrated circuit (ASIC), a field-programmable gate array (FPGA), or equivalent discrete or integrated logic circuitry. In some examples, the processor 80 may include multiple components, such as any combination of one or more microprocessors, one or more microcontrollers, one or more DSPs, one or more ASICs, or one or more FPGAs, as well as other discrete or integrated logic circuitry. The functions attributed to the processor 80 herein may be embodied as software, firmware, hardware or any combination thereof.

The signal generator 84 is electrically coupled to the electrodes 40, 42, 44, 46, 48, 50, 58, 62, 64, and 66, e.g., via conductors of the respective leads 18, 20, 22, or, in the case of the housing electrode 58, via an electrical conductor disposed within the housing 60 of the IMD 16. The signal generator 84 is configured to generate and deliver electrical stimulation therapy to the heart 12. For example, the signal generator 84 may deliver defibrillation shocks to the heart 12 via at least two electrodes 58, 62, 64, 66. The signal generator 84 may deliver pacing pulses via the ring electrodes 40, 44, 48 coupled to the leads 18, 20, and 22, respectively, and/or the helical electrodes 42, 46, and 50 of leads 18, 20, and 22, respectively. In some implementations, the signal generator 84 delivers pacing, cardioversion, or defibrillation stimulation in the form of electrical pulses. In other implementations, the signal generator 84 may deliver one or more of these types of stimulation in the form of other signals, such as sine waves, square waves, or other substantially continuous time signals.

The processor 80 controls the signal generator 84 to deliver stimulation therapy to the heart 12. The processor 80 may control the signal generator 84 to deliver stimulation according to a selected one or more therapy programs, which may be stored in memory 82. For example, the processor 80 may control the signal generator 84 to deliver electrical pulses with amplitudes, pulse widths, frequencies, or electrode polarities specified by the selected one or more therapy programs.

The processor 80 may select which of the electrodes 40, 42, 44, 46, 48, 50, 58, 62, 64, and 66 delivers electrical pulses. For example, the signal generator 84 may include a switch module that the processor 80 may use to select, e.g., via a data/address bus, which of the available electrodes are used to deliver pacing, cardioversion, or defibrillation pulses. The switch module may include a switch array, switch matrix, multiplexer, or any other type of switching device suitable to selectively couple electrical pulses to electrodes selected by the processor 80.

The electrical sensing module 86 monitors signals from at least one of the electrodes 40, 42, 44, 46, 48, 50, 58, 62, 64 or 66 in order to monitor electrical activity of the heart 12. The processor 80 may select which of the electrodes 40, 42, 44, 46, 48, 50, 58, 62, 64, and 66 function as sense electrodes. For example, the electrical sensing module 86 may include a switch module that the processor 80 may use to select, e.g., via a data/address bus, which of the electrodes are used to monitor electrical activity of the heart 12.

The electrical sensing module 86 may include multiple detection channels, each of which may comprise an amplifier. In response to the signals from processor 80, the switch module within the electrical sensing module 86 may couple selected electrodes to each of the detection channels.

The processor 80 may implement programmable counters. If the IMD 16 is configured to generate and deliver pacing pulses to the heart 12, such counters may control the basic time intervals associated with DDD, VVI, DVI, VDD, AAI, DDI, DDDR, VVIR, DVIR, VDDR, AAIR, DDIR and other modes of pacing. Intervals defined by the processor 80 may include atrial and ventricular pacing escape intervals, refractory periods during which sensed P-waves and R-waves are ineffective to restart timing of the escape intervals, and the pulse widths of the pacing pulses. The durations of these intervals may be determined by the processor 80 in response to stored data in memory 82.

A portion of memory 82 may be configured as a plurality of recirculating buffers, capable of holding a series of measured intervals, which may be analyzed by the processor 80 to determine whether the patient's heart 12 is presently exhibiting atrial or ventricular tachyarrhythmia. The processor 80 may detect tachyarrhythmia using any suitable tachyarrhythmia detection algorithm. In the event that the processor 80 detects an atrial or ventricular tachyarrhythmia, an anti-tachyarrhythmia pacing regimen, or cardioversion or defibrillation shock, may be loaded by the processor 80 and implemented using the signal generator 84.

The IMD 16 may comprise one or more sensors, such as sensor 87. The sensor 87 may comprise a chemical sensor, such as a glucose or pH sensor. The sensor 87 may comprise a pressure sensor (e.g., a capacitive sensor) that senses intracardiac or other cardiovascular pressure. The sensor 87 may comprise a motion sensor. The motion sensor may be, for example, an accelerometer or piezoelectric element. The sensor may also comprise a heart sound sensor, or any sensor capable of generating a signal that varies as a function of mechanical activity, e.g., contraction of the heart 12. The processor 80 may receive one or more signals from the sensor 87 or a plurality of sensors. The processor 80 may monitor, among other things, the mechanical activity of the heart 12 based on signals from the one or more sensors.

The sensor 87 may be positioned in various locations in the diagnostic system 10. For example, the sensor 87 may be located within the housing 60, outside of the housing 60, or on or within on or more of the leads 18, 20, 22. The sensor 87 may communicate with the IMD 16 via wireless communication when the sensor is located outside of the housing 60. In some implementations, the sensor 87 may be external (i.e., not implanted).

The communication module 88 includes any suitable hardware, firmware, software or any combination thereof for communicating with another device, such as the programmer 24. Under the control of the processor 80, the communication module 88 may receive downlink telemetry from and send uplink telemetry to the programmer 24 with the aid of an antenna (not shown), which may be internal and/or external. The processor 80 may provide the data to be uplinked to the programmer 24 and the control signals for telemetry circuitry within the communication module 88, e.g., via an address/data bus.

In some examples, the processor 80 may transmit atrial and ventricular heart signals detected by atrial and ventricular sense amplifier circuits within the electrical sensing module 86 to the programmer 24. The atrial and ventricular heart signals may be generally referred to as CEGM waveforms. In some cases, e.g., when detected via intracardiac electrodes, the CEGM waveforms may be IEGM waveforms. The programmer 24 may interrogate the IMD 16 to receive the CEGM waveforms. The processor 80 may also provide the CEGM waveforms to the programmer 24 in real-time via the communication module 88, e.g., when the electrical sensing module 86 senses the waveforms.

The processor 80 may store the CEGM waveforms in memory 82, and retrieve the stored CEGM waveforms from memory 82. The processor 80 may also generate and store marker channel data in memory 82. The marker channel data may indicate the occurrence and timing of sensing and therapy events, e.g., P-waves, R-waves, pacing pulses, anti-tachycardia pacing, cardioversion shocks, or defibrillation shocks, detected or undertaken by the IMD 16. The programmer 24 may interrogate the IMD 16, via the communication module 88, to receive the marker channel data. The processor 80 may also provide the marker channel data to the programmer 24 in real-time via the communication module 88, e.g., when the marker channel data is generated.

The processor 80 may store CEGM waveforms corresponding to physiological episodes, such as tachyarrhythmias, in memory 82. For example, processor 80 may store CEGM waveforms for atrial and ventricular tachycardia and fibrillation episodes, in response to the detection of the tachycardia or fibrillation. The processor 80 may also store CEGM waveforms corresponding to nonsustained tachycardia (NST) in memory 82 in response to detection of the NST using any suitable NST detection technique. The programmer 24 may interrogate the IMD 16, via the communication module 88, to receive the stored CEGM waveforms.

The processor 80 may also store parametric data in memory 82. Parametric data may include, for example, impedance measurements, trends of impedance measurements, or statistical or other processed values determined based on impedance measurements. Other parametric data may include data indicating the current status of a power source 98 of IMD 16. The programmer 24 may interrogate the IMD 16, via the communication module 88, to receive the parametric data. The processor 80 may also provide the parametric data to the programmer 24 in real-time via the communication module 88, e.g., when the parametric data is measured.

The processor 80 may also store other sensed physiological parameters of the patient 14, such as intracardiac or intravascular pressure, activity, posture, respiration, thoracic impedance, blood glucose levels, or pH levels, which may be sensed via one or more sensors 87, for example. The programmer 24 may interrogate the IMD 16, via the communication module 88, to receive the other sensed physiological parameters. The processor 80 may also provide the other sensed physiological parameters to the programmer 24 in real-time via the communication module 88, e.g., when sensed physiological parameters are measured.

The various components of the IMD 16 are coupled to a power source 98, which may include a rechargeable or non-rechargeable battery. A non-rechargeable battery may be capable of holding a charge for several years, while a rechargeable battery may be inductively charged from an external device, e.g., on a daily or weekly basis.

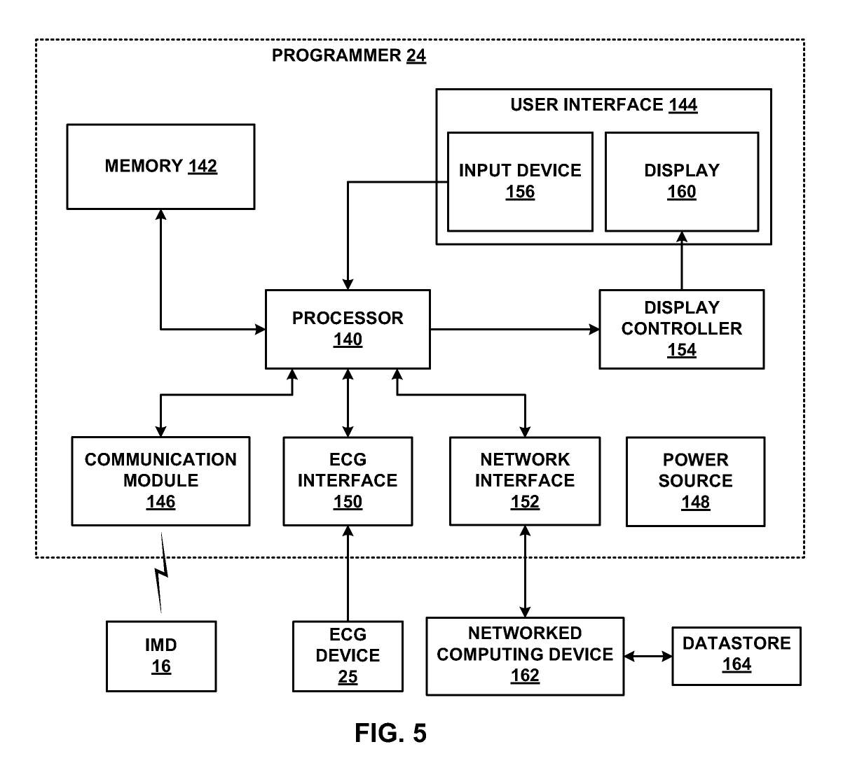

FIG. 5 is an example functional block diagram of the programmer 24. The programmer 24 includes a processor 140, memory 142, a user interface 144, a communication module 146, a power source 148, an ECG interface 150, a network interface 152, and a display controller 154. The programmer 24 may be a dedicated hardware device with dedicated software for communicating with the IMD 16. For example, the programmer 24 may be a dedicated hardware device that programs operational parameters of the IMD 16 and/or receives data from the IMD 16. Alternatively, the programmer 24 may be an off-the-shelf computing device, such as a desktop or laptop computer, running an application that enables the programmer 24 to communicate with the IMD 16 (e.g., receive data from the IMD 16 and/or program the IMD 16). Accordingly, the programmer 24 represents any computing device capable of performing the functions attributed to the programmer 24 in the present disclosure. In some implementations, components of the programmer 24 may be housed in a single housing, for example, a molded plastic casing. For example, the processor 140, memory 142, the user interface 144, the communication module 146, the ECG interface 150, the network interface 152, and the display controller 154 may be housed in the housing.

The user interacts with the programmer 24 using the user interface 144. The user interface 144 includes an input device 156 and a display 160. The user enters data into the programmer 24 using the input device 156. The input device 156 may include various devices for entering data. The input device 156 may include a keypad, for example, an alphanumeric keypad or a reduced set of keys associated with particular functions of the programmer 24.

The input device 156 may also include a freehand peripheral input device. A freehand peripheral input device may include device that allow the user to move a cursor using a freehand movement. Freehand input devices may include, but are not limited to, a mouse, a stylus, and a touchscreen (e.g., that detects a finger or other object).

The user may make discrete selections on the display 160 using the freehand peripheral device. For example, the user may use the mouse to click discrete controls on the display to activate various functions and options of the programmer 24 to be discussed herein. The user may also use at least one of the mouse, the stylus, and the touchscreen to annotate the display 160. Annotations may include illustrations on various sections of the display 160. For example, the user may use the stylus to draw an illustration on a waveform displayed on the display 160.

The network interface 152 may communicate with a networked computing device 162, for example, an off-the-shelf desktop/laptop or server computer. Accordingly, the user may interact with the programmer 24 and/or the IMD 16 remotely using the networked computing device 162. The networked computing device 162 may include a display that displays similar information as the display 160. The networked computing device 162 may also include an input device having similar functionality as the input device 156. Accordingly, a user may control the programmer 24 and view information displayed on the programmer 24 using the networked computing device 162.

The display 160 may include a cathode ray tube (CRT) display or a liquid crystal display (LCD), for example. In some implementations, the display 160 may include a touchscreen. Accordingly, the input device 156 may be integrated into the display 160 in some implementations.

The processor 140 can take the form of one or more microprocessors, DSPs, ASICs, FPGAs, programmable logic circuitry, or the like, and the functions attributed to the processor 140 herein may be embodied as hardware, firmware, software or any combination thereof. The processor 140 of the programmer 24 may provide any of the functionality ascribed herein, or otherwise perform any of the methods described herein. For example, the processor 140 may provide for the measurement function, the freehand annotation function, the typing annotation function, the attachment annotation function, the searching function, the comparison function, the timing diagram function, and the medical record generation function as ascribed to the programmer 24 hereinafter.

Memory 142 may store instructions that cause the processor 140 to provide the functionality ascribed to the programmer 24 herein, and information used by the processor 140 to provide the functionality ascribed to the programmer 24 herein. Memory 142 may include any fixed or removable magnetic, optical, or electrical media, such as RAM, ROM, CD-ROM, hard or floppy magnetic disks, EEPROM, or the like. Memory 142 may also include a removable memory portion that may be used to provide memory updates or increases in memory capacities. A removable memory may also allow patient data to be easily transferred to another computing device, or to be removed before the programmer 24 is used to program therapy for another patient. Memory 142 may also store information that controls therapy delivery by the IMD 16, such as stimulation parameter values.

In some implementations, the programmer 24 may store data in a networked datastore 164. For example, the processor 140 may transfer data to the datastore 164 using the network interface 152. Accordingly, in some examples, the networked computing device 162 may be a server that communicates with the programmer 24 to store data from programmer 24 in the datastore 164 and retrieve data from the datastore 164 for use by the programmer 24. In some implementations, the networked computing device 162 and the datastore 164 may represent general network technology and functionality similar to that provided by the Medtronic CareLink® Network developed by Medtronic, Inc., of Minneapolis, Minn. The data stored in the datastore 164 may include, for example, CEGM waveforms, marker channel data, ECG waveforms, and other data. In other implementations, the networked computing device 162 and the datastore 164 may represent or interface with a system configured to store electronic medical records, which may additionally or alternatively include other waveforms or medical information for the patient 14.

The programmer 24 may communicate wirelessly with the IMD 16, such as using RF communication or proximal inductive interaction. This wireless communication is possible through the use of the communication module 146, which may be coupled to an internal antenna or an external antenna. For example, an external antenna may be included in a programming head (not shown) that is coupled to the programmer 24. The programming head may be placed over heart 12 (i.e., IMD 16), as described above with reference to FIG. 1 to communicate with the IMD 16. The communication module 146 may include similar functionality as the communication module 88 of the IMD 16.

The communication module 142 may also be configured to communicate with another computing device via wireless communication techniques. Examples of local wireless communication techniques that may be employed to facilitate communication between programmer 24 and another computing device using the communication module 142 include RF communication according to the 802.11 or Bluetooth specification sets, infrared communication, e.g., according to the IrDA standard, or other standard or proprietary telemetry protocols. In this manner, other external devices may be capable of communicating with the programmer 24 without needing to establish a secure wireless connection.

The programmer 24 receives ECG waveforms via the ECG interface 150. For example, the ECG interface 150 may receive ECG waveforms from the ECG electrodes connected to the patient 14, or from the ECG device 25. Accordingly, in some implementations, ECG electrodes may be connected directly to the ECG interface 150 of the programmer 24. The processor 140 stores the ECG waveforms in the memory 142.

Although the programmer 24 is described as including the communication module 146 that wirelessly receives CEGM waveforms and the ECG interface 150 that receives ECG waveforms, in other implementations, the programmer 24 may include additional or alternative interfaces that receive additional or alternative waveforms. For example, the programmer 24 may include an interface that receives neurological waveforms, such as electroencephalography (EEG) waveforms or other sensed physiological waveforms indicating one of intracardiac or intravascular pressure, activity, posture, respiration, thoracic impedance, blood glucose levels, or pH levels, as examples. Accordingly, in some implementations, the processor 140 may store neurological waveforms or other sensed physiological waveforms in the memory 142. The systems and methods of the present disclosure may also be applicable to other types of acquired waveforms instead of or in addition to the aforementioned waveforms.

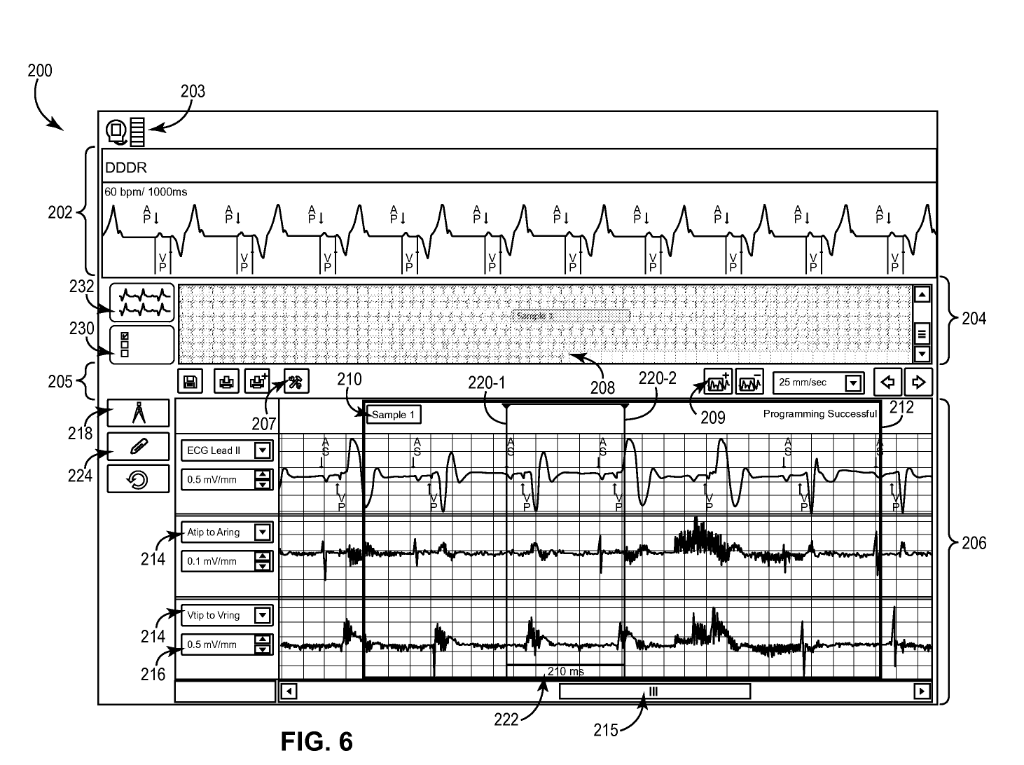

The display controller 154 receives data from the processor 140 and generates output images on the display 160. In other words, the display controller 154 is configured to display information on the display 160. For example, the display controller 154 may display CEGM waveforms, ECG waveforms, etc. Display of the CEGM and ECG waveforms will be discussed hereinafter in FIGS. 6-8

The power source 148 delivers operating power to the components of the programmer 24. The power source 148 may include a battery and/or adapter for connection to an alternating current (AC) wall socket.

In summary, the example programmer 24 of the present disclosure may receive data from the IMD 16 and the ECG device 25. Data received from the IMD 16 may include, but is not limited to CEGM waveforms and marker channel data. Data received from the ECG 25 may include but is not limited to, ECG waveforms. The processor 140 may store the CEGM waveforms and the marker channel data in memory 142 when the communication module 146 receives the CEGM waveforms and the marker channel data. The processor 140 may store the ECG waveforms in memory 142 when the ECG interface 150 receives the ECG waveforms.

The processor 140 may transfer the CEGM waveforms and the marker channel data to the display controller 154 to display the CEGM waveforms and the marker channel data as the CEGM waveforms and the marker channel data are received from the IMD 16. The processor 140 may also transfer the ECG waveforms to the display controller 154 to display the ECG waveforms as the ECG waveforms are received from the ECG interface 150. In other words, the programmer 24 may display received CEGM waveforms, marker channel data, ECG waveforms, and other data in real-time as the data is being received.

The processor 140 may transfer the CEGM waveforms, the marker channel data, the ECG waveforms, and other data to both the memory 142 and the display controller 154 concurrently. In other words, the CEGM waveforms, the marker channel data, the ECG waveforms, and other data viewed on the display 160 in real-time may also be stored in real-time. Additionally, as discussed hereinafter, the programmer 24 may also display stored CEGM waveforms, stored marker channel data, stored ECG waveforms, and other stored data selectively in response to a selection command input by the user. For example, in response to the selection command, the programmer 24 may display both real-time waveforms and stored waveforms while concurrently storing waveforms received from the communication module 146 and the ECG interface 150.

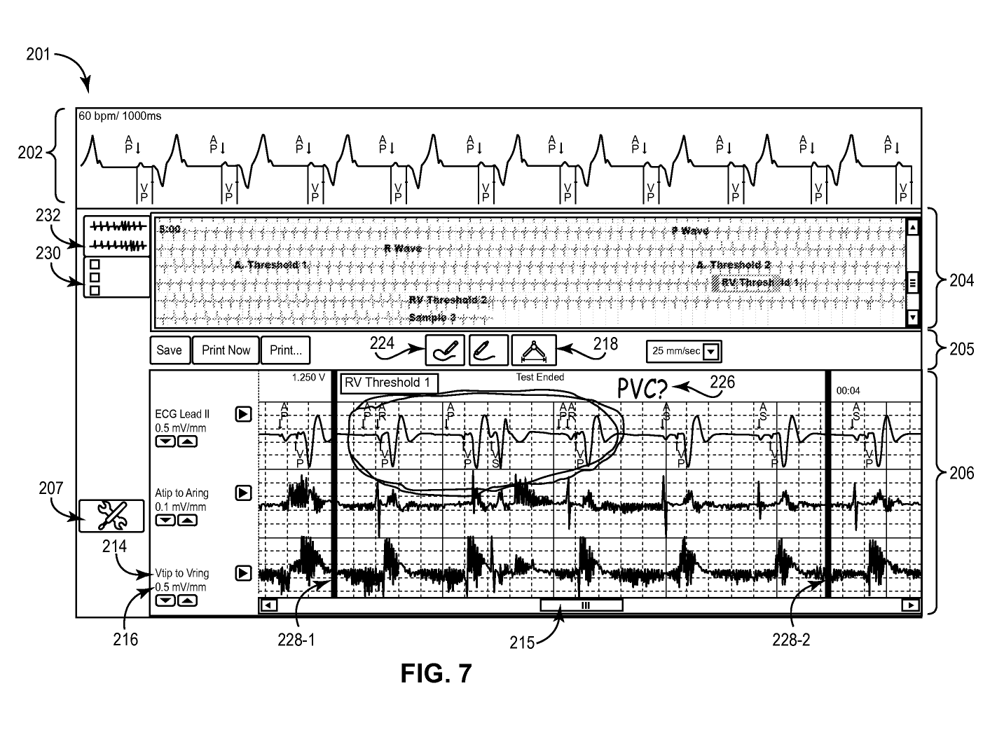

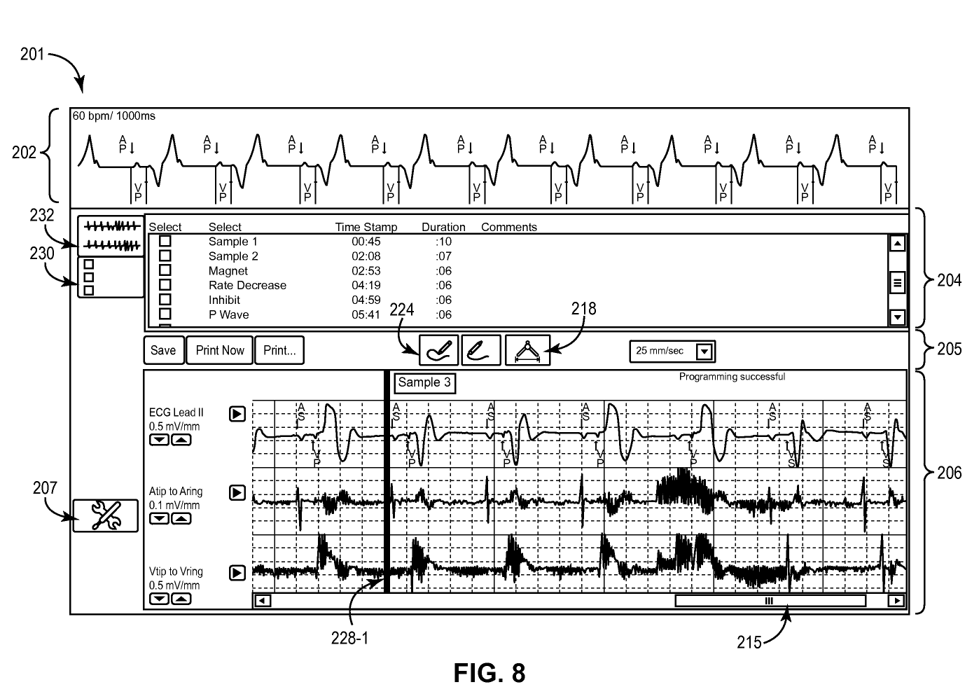

The programmer 24 of the present disclosure includes various functions for analyzing and reviewing data received from the IMD 16 and the ECG device 25. The user may access the various functions of the programmer 24 using the user interface 144. Accordingly, the various functions of the programmer 24 will now be discussed in conjunction with figures FIGS. 6-8, which illustrate the user interface. Two different example formats (i.e., graphical styles) of a graphic-user interface (GUI) are shown in FIGS. 6-8. A first format is shown in FIG. 6. A second format is shown in FIGS. 7-8. Although two separate formats are shown, the GUIs shown in FIGS. 6-8 allow the user to access similar functions of the programmer 24.

In general, the user interface 144 displays information to the user related to the CEGM waveforms and ECG waveforms currently being received as well as the CEGM and ECG waveforms that are stored in memory 142. Using the input device 156, the user may access the various functions of the programmer 24 to review and analyze the CEGM and ECG waveforms currently being received and also the CEGM and ECG waveforms stored in the memory 142. Functions of the programmer to be discussed herein include, but are not limited, to a measurement function, a freehand annotation function, a typing annotation function, an attachment annotation function, a searching function, a waveform comparison function, a timing diagram function, and a medical record generation function. The example user interface 144 and each of the above example functions of the programmer 24 are now discussed in turn.

FIG. 6 shows a first example GUI 200 displayed on the display 160. The GUI 200 of FIG. 6 includes 3 sections for displaying waveforms: a real-time section 202, a stored-data section 204, and a reviewing section 206. The real-time section 202 may display data as the data is being received. For example, the real-time section 202 may display CEGM waveforms, ECG waveforms, marker channel data, and/or other data as the waveforms and data are being received. In other words, the real-time section 202 may display the data received from the IMD 16 and the ECG device 25 in real-time.

The real-time section 202 in FIG. 6 displays an ECG waveform and marker channel data as the ECG waveform and marker channel data are being received. “DDDR” in the upper left of the display indicates the mode of the IMD 16. 60 bpm/1000 ms may indicate the heart rate. AP and VP may indicate delivery of atrial and ventricular pacing by the IMD 16, respectively.

Although the ECG waveform is displayed in real-time in the real-time section 202 in FIG. 6, waveforms from other devices connected to the programmer 24, e.g., CEGM waveforms from the IMD 16, may also be displayed in the real-time section 202. Although a single waveform is displayed in the real-time section 202 in FIG. 6, more than one waveform may be displayed in the real-time section 202.

The stored-data section 204 may display a history of the waveforms received from the IMD 16 and/or the ECG device 25. In other words, the stored-data section 204 displays the stored waveform or waveforms from the time the waveforms are initially received by the programmer 24 up until the most current waveform received. In the illustrated example, the stored-data section 204 illustrates the history of the ECG waveforms received from the IMD 16, but in other examples may additionally or alternatively illustrate the history of CEGM or other received waveforms. Specifically, the upper left corner of the stored-data section 204 indicates the earliest stored ECG waveform, while the most current data is illustrated at 208. The most current waveform illustrated at 208 may correspond to the waveform shown in the real-time section 202.

The most current data may continue to fill the stored-data section 204 from left to right and from top to bottom of the stored-data section 204. Accordingly, the stored-data section 204 may show all of the stored waveform, starting from the upper left of the stored-data section 204 and moving to the lower right of the stored-data section 204, skipping to a new line at the right side of the stored-data section 204. A scroll bar illustrated on the right side of the stored-data section 204 may be used to scroll up and down in order to bring data into view that has been collected at different times. For example, scrolling up brings older data into view, while scrolling down brings more recent data into view. The user may use the input device 156 (e.g., a mouse) to activate the scroll bar in order to scroll through and view stored data in the stored-data section 204.

Accordingly, using the real-time section 202 and the stored-data section 204 of the display 160, the user may view waveforms that are acquired in real-time by the communication module 146 or the ECG interface 150 along with a history of the corresponding waveform that is stored in the memory 142. For example, in FIG. 6, the user may view an ECG waveform in real-time in the real-time section 202 while viewing the history of previously acquired ECG waveforms in the stored-data section 204.

The reviewing section 206 may display a selected portion of the stored CEGM and ECG waveforms. For example, the user may select any portion of the waveform, e.g., ECG, illustrated in the stored-data section 204 for viewing the time-corresponding portions of one or more waveforms or data, e.g., ECG, CEGM and marker channel data, in the reviewing section 206. Selection of a portion of the stored CEGM and ECG waveforms may be referred to as a “selection command.” For example, a selection command may be made by clicking, with a mouse, on a selected portion of the stored waveform in the stored-data section 204.

The reviewing section 206 of FIG. 6 illustrates a portion of the stored CEGM and ECG waveforms selected from the stored-data section 204. Specifically, most of the reviewing section 206 in FIG. 6 shows a region of the stored waveforms referred to as “Sample 1” as indicated at 210 and bounded by a box indicated at 212. “Sample 1” is a portion of the stored CEGM and ECG waveforms selected from the stored-data section 204. Specifically, the box labeled “Sample 1” in the reviewing section 206 corresponds to the text box of the stored-data section 204 that includes the text “Sample 1.” The text box including “Sample 1” in the stored-data section 204 is an example of a waveform segment that has been annotated using an annotation function of the programmer 24, to be discussed herein. The bounds of the text box indicate the length of the waveform segment associated with the text box. In some implementations, the text box may include highlighting.

Waveform selection menus (e.g., illustrated at 214) at the left of the reviewing section 206 indicate the specific waveform being viewed in the reviewing section 206. For example, in FIG. 6, the bottom third of the reviewing section 206 displays a CEGM waveform indicating a voltage difference between electrodes 42 (Vtip) and 40 (Vring). Accordingly, the waveform above the “Vtip to Vring” waveform illustrates a voltage difference between electrode 50 (Atip) and electrode 48 (Aring). The waveform above the “Atip to Aring” waveform illustrates a voltage detected by a lead (i.e., lead II) connected to the ECG device 25. Each waveform in the reviewing section 206 also includes corresponding axis scales. For example, the magnitude axis of “Vtip to Vring” is 0.5 mV/mm, as indicated at 216.

Although the reviewing section 206 has been described as displaying the CEGM waveforms and the ECG waveforms that are stored in the memory 142, the reviewing section 206 may also display real-time waveforms and data as the waveforms and data are received. For example, the reviewing section 206 may display CEGM and ECG waveforms and marker channel data as the communication module 146 and the ECG interface 150 receive the waveforms and data.

The user may selectively view different portions of the waveform in the reviewing section 206. In other words, the user may scan a waveform displayed in the reviewing section 206. For example, the user may view the waveform in real-time in the reviewing section 206, and may also scan backward in time to review saved portions of the waveform. Subsequent to scanning backward in time, the user may scan forward in time to view the portion of the waveform that is being collected in real-time. The user may use a scroll bar 215 to scan forward and backward in time in the reviewing section 206. Moving the scroll bar 215 to the right may allow the user to view portions of the waveform that were stored more recently. Moving the scroll bar 215 to the left may allow the user to view older portions of the waveform. Accordingly, moving the scroll bar 215 to a furthest position to the right may allow the user to view real-time data as the waveform is being received, while moving the scroll bar 215 to a leftmost position may allow the user to view the first saved portions of the waveform. Although the scroll bar 215 is illustrated as a control used to scan the waveform in the reviewing section 206, other controls may be used to scan the waveform, such as buttons (e.g., fast forward and/or rewind buttons).

Although the reviewing section 206 includes 3 waveforms, the reviewing section 206 may include more or less than three waveforms. For example, the reviewing section 206 may include additional waveforms generated by the ECG device 25 and additional voltage measurements made by the IMD 16 based on different electrode combinations. In some implementations, the additional waveforms may be selected using the selection menus 214. For example, the input device 156 (e.g., a mouse) may be used to activate the selection menu 214 and expose other electrode combinations. Specifically, clicking on the selection menu including the text “ECG Lead II” may expose a list of other ECG electrode voltages which may be displayed in the reviewing section 206.

The programmer 24 includes a measurement function that allows the user to measure the waveforms in the reviewing section 206. Accordingly, the programmer 24 allows for measurement of the real-time waveforms and/or the stored waveforms and displays the measurements taken on the display 160.

The user may select a measurement button 218 on the user interface 144 using the input device 156 to activate the measurement function of the programmer 24. The measurement function may display a magnitude of the waveforms in the reviewing section 206. The measurement function may also display an amount of time between two points in the reviewing section 206. For example, measurement bars 220-1 and 220-2 may be selectively moved by the user across the reviewing section 206 to different points of interest, while the time indicator 222 along the bottom of the reviewing section 206 indicates the amount of time (in ms) between the two measurement bars 220-1 and 220-2. In some implementations, for each waveform, a voltage corresponding to the locations of the measurement bars 220-1 and 220-2 may be displayed, along with the difference between the voltages at the locations of the measurements bars 220-1, 220-2. Measurements made by the measurement function may be displayed, for example, in the reviewing section 206.

The user may select a freehand annotation button 224 to activate a freehand annotation function of the programmer 24. The freehand annotation function allows the user to draw on the display 160 using freehand motion. For example, the user may use a mouse, a stylus, or a touchscreen to enter the freehand annotations. The user may annotate the waveforms in the reviewing section 206 using the freehand annotation function. For example, the user may annotate stored waveforms and/or real-time waveforms.

A second example GUI 201 in FIG. 7 shows a freehand annotation in the reviewing section 206, as illustrated at 226. The freehand annotation includes the text “PVC?” next to a freehand circle around the ECG Lead II waveform. For example, the freehand annotation “PVC?” may indicate that the circled area of interest may correspond to a premature ventricular contraction. The freehand annotations may be stored along with the waveforms in memory 142 and later recalled to be displayed with the waveforms.

The programmer 24 may include a typing annotation function. The user may enter typed (e.g., alphanumeric) annotations on the display 160 using the input device 156 (e.g., a keyboard) of the programmer 24. Referring back to FIG. 6, the stored-data section 204 includes a text box having the text “Sample 1.” The text box including “Sample 1” is an example of the typing annotation function of the programmer 24. For example, the user may select a segment of the waveform displayed in the stored-data section 204 and enter the text “Sample 1” using the input device 156 to create the text box associated with that waveform segment. Typed annotations associated with a waveform segment may operate as a “title” of the segment. Accordingly, the waveform segment in the stored-data section 204 of FIG. 6 may have the title “Sample 1.” A waveform may be searched for based on the associated title using a searching function of the programmer 24.

Typed annotations may also include “tick marks” (not shown) associated with various points of the stored waveforms. For example, the user may have a push button on the input device 156 that may be pressed to create a tick mark on the waveform. Tick marks may be indicated in the real-time section 202, the stored-data section 204, and/or the reviewing section 206. Accordingly, when a user sees an area of interest on the real-time section 202 of the display 160, the user may depress the button on the input device 156 and create a tick mark that marks the area of interest. The user may later search for the tick mark using a searching function, or the user may visually search the stored-data section 204 for the tick mark and then review the waveform segment including the tick mark in the reviewing section 206.

Referring to FIG. 7, a plurality of typed annotations, i.e., waveform segment titles, are added in the stored-data section 204. For example, the annotations in FIG. 7 include: 5:00, P Wave, R Wave, and A. Threshold 1, and RV Threshold 1. The user may select the typed annotations, e.g., using a mouse click, to view the annotated waveform segments in the reviewing section 206. For example, the reviewing section 206 of FIG. 7 shows the annotated waveform segment labeled “RV Threshold 1.” The start and end of “RV Threshold 1” is indicated by the markers at 228-1 and 228-2.

The programmer 24 may include an attachment annotation function. The attachment annotation function may allow for files of various formats to be stored in the memory 142 and associated with a waveform segment. For example, attachments may include image files, such as a magnetic resonance imaging file. Attachments may also include audio files, such as voice annotations from a microphone when the input device 156 includes a microphone. Accordingly, in some implementations, the user may attach a file (e.g., image/video and/or audio) to a selected waveform segment and later recall the attached file when reviewing the waveform segment in the reviewing section 206.

The annotation functions described above (i.e., freehand, typed, and attachment) may be manually entered by the user in the real-time section 202, the stored-data section 204, and the reviewing section 206. In addition to the annotation functions discussed above, the programmer 24 and/or the IMD 16 may annotate the waveforms automatically. For example, the IMD 16 may generate marker channel data and transmit the marker channel data to the programmer 24. The programmer 24 may then add the marker channel data to the CEGM and ECG waveforms in real-time and store the marker channel data in memory 142. Additionally or alternatively, the programmer 24 may automatically generate annotations. For example, the annotations of the P Wave and R Wave of FIG. 7 may be automatic annotations inserted by the programmer 24 in response to detection of a P wave and an R wave, respectively, by the programmer 24. As a further example, the GUIs 200 and 201 include annotations that may be automatically added by the programmer 24. Specifically, the annotation in the reviewing section 206 (i.e., “programming successful”) may have been added by the programmer 24 automatically to indicate that the programmer 24 had successfully programmed the IMD 16.

The programmer 24 includes a searching function. The searching function may search for waveform segments based on the title associated with the waveform segment. Referring to FIG. 8, the stored-data section 204 illustrates an example searching function of the programmer 24. Specifically, the stored-data section 204 lists titles of stored waveform segments, time stamps associated with the stored waveform segments, and durations of the stored waveform segments. The searching function may be called by pressing a searching button 230, for example. The stored-data section 204 that displays the waveform history, as shown in FIGS. 6-7 may be recalled by pressing button 232. Accordingly, the buttons 230 and 232 may be pressed to toggle between the searching function and viewing of the stored waveform.

The title of the waveform segments shown in FIG. 8 includes: Sample 1, Sample 2, Magnet, Rate Decrease, etc. Timestamps are associated with each of the waveform segments. The time stamps may indicate a time at which the waveform segment occurs in the stored waveform. Durations (e.g., :10, :07, :06, etc.) are associated with each of the waveform segments. The durations may indicate a length of the waveform segments.

The user may use the searching function of the programmer 24 to organize the waveform segments, for example, alphabetically by title, chronologically according to time stamp, or by total duration of the waveform segments. Additionally, a search box is contemplated as part of the searching function of the programmer 24. For example, the user may enter text into the search box to search for titles of stored waveform segments. Additionally, parameters of the stored waveform segments may be searchable. For example, the user may search stored waveform segments based on intrinsic rate present in the waveform segments, or based on which waveform segments include voltages that are greater than a threshold voltage.

The programmer 24 includes a comparison function that allows the user to compare two separate waveforms on the display 160, for example, in the reviewing section 206. Using the comparison function, the user may compare any combination of stored waveforms and real-time waveforms. For example, in some implementations, the user may compare two stored waveform segments in the reviewing section 206 by selecting two waveform segments from the stored-data section 204. In other words, the user may compare two separate portions of the stored ECG waveform, as selected from the stored-data section 204. In other implementations, the user may select a stored waveform segment from the stored-data section 204 and compare, in the reviewing section 206, the stored waveform segment to the real-time waveform currently being received.

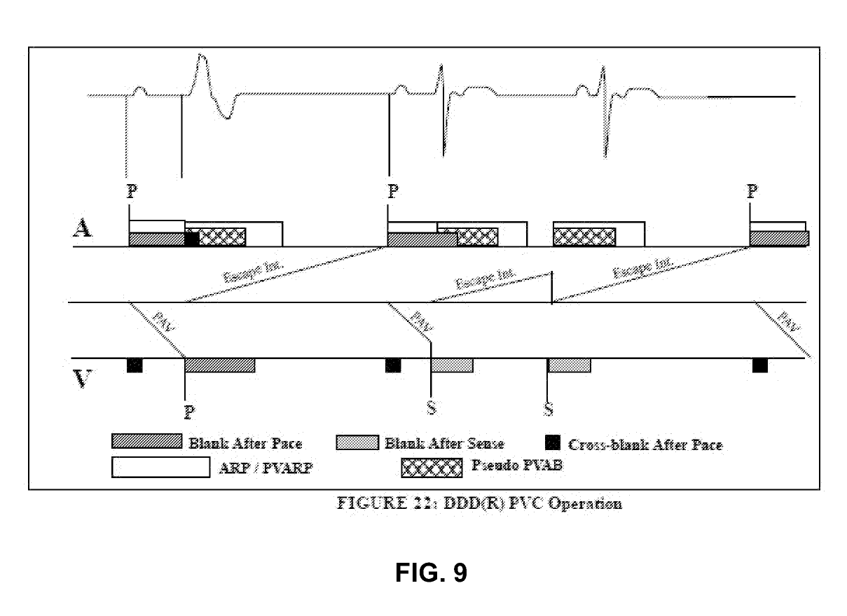

The programmer 24 may include a timing diagram function that displays a timing diagram that may be used to explain pacing behavior in complex interactions with unusual physiologic rhythms. The timing diagram may also be used to analyze how IMD 16 interprets physiological signals. The timing diagram may show information related to why the IMD 16 is pacing or what IMD 16 is sensing. In some examples, the timing diagram may include one or more electrogram waveforms in conjunction with marker channel data. The timing diagram may also give information related to why a pace did or did not occur. FIG. 9 shows an example timing diagram illustrating pace timing with a PVC. The timing diagram function may display the example timing diagram of FIG. 9 in the reviewing section 206, for example.

The programmer 24 may include an electronic medical record (EMR) generation function. For example, the programmer 24 may format the CEGM waveforms, the ECG waveforms, and the marker channel data into a format that may be stored in an EMR database. The programmer 24 may upload the formatted data, via the network interface 152 to an EMR database.

Although the programmer 24 is described as storing waveforms in memory 142 and displaying stored waveforms from the memory 142, the programmer 24 may also store and retrieve waveforms from the datastore 164. Accordingly, the datastore 164 may operate as a database of stored waveforms that may be viewed using the programmer 24.

The GUIs 200 and 201 may include additional features not discussed above that provide additional functionality and convenience to the user. For example, the GUI 200 includes a telemetry strength indicator 203 that may indicate a quality of the communication channel between the IMD 16 and the programmer 24. The GUI 201 includes an erase button adjacent to the freehand annotation button 224 that allows the user to erase freehand annotations added to the waveforms. The GUIs 200 and 201 include a toolbar section 205 that includes a feed rate (e.g., 25 mm/sec) of the virtual strip chart. The toolbar section 205 also includes other functionality such as buttons that allow saving and printing of the waveforms. The GUIs 200 and 201 also include preference buttons 207 which allow for selection of user preferences, such as the number of waveforms to display in the reviewing section 206, among other preferences. The GUI 200 includes magnification buttons 209 that may be used to zoom in and zoom out on the waveforms in the reviewing section 206 and/or the stored-data section 204.

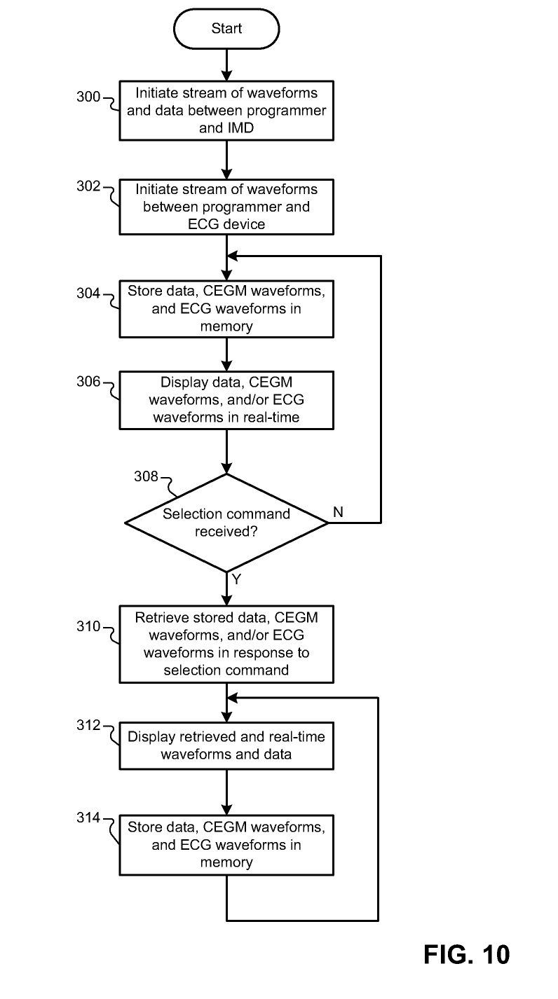

Referring to FIG. 10, a method for reviewing and analyzing electrogram data according to the present disclosure is shown. Communication between the programmer 24 and the IMD 16 is initiated (300). Specifically, the communication module 146 of the programmer 24 begins to receive marker channel data and a stream of the CEGM waveforms from the IMD 16. Communication between the programmer 24 and the ECG device 25 is initiated (302). Specifically, the ECG interface 150 of the programmer 24 begins to receive a stream of ECG waveforms from the ECG device 25. The processor 140 stores the marker channel data and the CEGM and ECG waveforms in memory 142 (304). The display controller 154 displays the marker channel data, the CEGM waveforms, and/or the ECG waveforms as the waveforms and marker channel data are received by the programmer 24 (306). In other words, the display controller 154 displays the marker channel data and the waveforms in real-time on the display 160 while the processor 24 is concurrently storing the waveforms and the marker channel data in memory 142.

The processor 140 determines whether a selection command has been received from the input device 156 (308). If the processor 140 does not receive the selection command from the input device 156, the processor 140 continues to store the marker channel data and the CEGM and ECG waveforms (304) and the display controller 154 continues to display the marker channel data, the CEGM waveforms, and/or the ECG waveforms in real-time (306). If the processor 140 receives the selection command from the input device 156, the processor 140 retrieves the stored marker channel data, the CEGM waveforms, and/or the ECG waveforms from memory 142 that correspond to the selection command (310). In other words, a user may enter a selection command in the input device 156 that indicates a desire to view a specific portion of a stored waveform. In response to the selection command, the processor 140 retrieves the specific portion of the stored waveform and marker channel data from memory 142. The display controller 154 displays the specific portion of the waveform and marker channel data retrieved from memory 142 and also displays the real-time marker channel data, the CEGM waveforms, and/or the ECG waveforms as the marker channel data, the CEGM waveforms, and/or the ECG waveforms are received (312). The processor 140 continues to store the marker channel data, the CEGM waveforms, and the ECG waveforms in memory 142 while the display controller 154 is displaying both the specific portion of the waveform and marker channel data retrieved from memory 142 and the real-time waveforms and marker channel data (314).

Although the disclosure is described with respect to an implantable cardiac device, the techniques described may be applicable to other implantable devices, such as devices that provide spinal cord stimulation, deep brain stimulation, pelvic floor stimulation, gastric stimulation, occipital stimulation, functional electrical stimulation, and the like. Accordingly, the techniques described herein may be applicable to non-cardiac signals received from electrodes located on or within a patient.

Furthermore, the techniques of the present disclosure may be applicable to implantable medical devices that do not deliver therapy, such as implantable monitors. For example, the techniques of the disclosure may be applicable to insertable cardiac monitors that monitor heart rhythms and record ECGs, which may be associated with physiological events, e.g., fainting.

The techniques of the present disclosure may also be applicable to signals received from sensors. In other words, the programmer 24 may display signals received from sensors (e.g., the sensor 87) that measure physiological characteristics other than EGM waveforms. For example, the sensors may include a cardiovascular pressure sensor, an accelerometer, an oxygen sensor, a sensor that measures transthoracic impedance, or a glucose monitor. The programmer 24 may display signals received from the sensors in a similar manner as the programmer 24 displays EGM waveforms. For example, the programmer 24 may display data received from the sensors in real-time in the real-time section 202. The programmer 24 may also display stored data from the sensors in the stored-data section 204. Additionally, the user may select portions of the stored data from the sensors in the stored-data section 204 to display in the reviewing section 206 and may scan sensor waveforms in the reviewing section 206 using the scroll bar 215.

The techniques described in this disclosure, including those attributed to the IMD 16, programmer 24, or various constituent components, may be implemented, at least in part, in hardware, software, firmware or any combination thereof. For example, various aspects of the techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as random access memory (RAM), read-only memory (ROM), non-volatile random access memory (NVRAM), electrically erasable programmable read-only memory (EEPROM), FLASH memory, magnetic data storage media, optical data storage media, or the like. The instructions may be executed to support one or more aspects of the functionality described in this disclosure.

Various examples have been described. These and other examples are within the scope of the following claims.

1. A computing device comprising:
a memory device;
a communication module configured to wirelessly receive a stream of first electrogram (EGM) waveforms of a patient from an implantable medical device (IMD);
an interface configured to receive a stream of second EGM waveforms of the patient that are based on external measurement of changes in electrical potential associated with the patient;
a processor configured to store the first and second EGM waveforms in the memory device and retrieve the stored first and second EGM waveforms from the memory device;
a display controller configured to display the first and second EGM waveforms together on a display when the first and second EGM waveforms are received; and
an input device configured to receive a selection command from a user, wherein the processor retrieves a portion of at least one of the first and second stored EGM waveforms from the memory device in response to the selection command, and wherein the display controller displays the retrieved portion of the at least one of the first and second stored EGM waveforms on the display while the processor concurrently stores the streams of the first and second EGM waveforms in the memory device.

2. The computing device of claim 1, wherein the first EGM waveforms comprise cardiac electrogram (CEGM) waveforms from the IMD, and the second EGM waveforms comprise electrocardiogram (ECG) waveforms.

3. The computing device of claim 2, wherein the IMD includes at least one of a cardiac pacemaker or a cardioverter-defibrillator.

4. The computing device of claim 2, wherein the CEGM waveforms include intracardiac waveforms.

5. The computing device of claim 1,
wherein the communication module is configured to wirelessly receive marker channel data from the IMD that indicates the occurrence and timing of therapy and sensing events associated with the IMD,
wherein the processor is configured to store the marker channel data in the memory device and retrieve the stored marker channel data from the memory device,
wherein the display controller is configured to display the marker channel data on the display together with the first and second EGM waveforms when the marker channel data is received,
wherein the processor retrieves a portion of the marker channel data from the memory device in response to the selection command, and
wherein the display controller displays the retrieved portion of the marker channel data on the display while the processor concurrently stores received marker channel data in the memory device.

6. The computing device of claim 1, wherein the processor is configured to set operational parameters of the IMD via the communication module.

7. The computing device of claim 1, further comprising a housing, wherein the housing includes the memory device, the communication module, the interface, the processor, and the display controller.

8. The computing device of claim 1, wherein the input device is configured to receive an annotation from the user, wherein the display controller displays the annotation on at least one of the first and second EGM waveforms displayed while the processor concurrently stores the streams of first and second waveforms in the memory device.

9. The computing device of claim 8, wherein the input device includes an alphanumeric keypad, and wherein the annotation includes an alphanumeric annotation.

10. The computing device of claim 8, wherein the input device includes a freehand input device, and wherein the annotation is a freehand annotation.

11. The computing device of claim 10, wherein the freehand input device includes at least one of a mouse, a stylus, and a touchscreen.

12. The computing device of claim 8, wherein the processor is configured to associate the annotation with at least one of the first and second stored EGM waveforms, and wherein the processor is configured to search for the at least one of the first and second stored EGM waveforms based on the annotation.

13. The computing device of claim 1, wherein the display controller is configured to concurrently display a first segment of one of the first and second EGM waveforms and a second segment of the one of the first and second EGM waveforms.

14. The computing device of claim 13, wherein each of the first and second segments is a stored segment.

15. The computing device of claim 13, wherein the first segment is a stored segment retrieved from the memory device and the second segment is a currently received segment.

16. The computing device of claim 1, further comprising a network interface configured to transfer data to an electronic medical record (EMR) database, wherein the processor is configured to transfer at least one of the first and second stored EGM waveforms to the EMR database using the network interface.

17. The computing device of claim 1, wherein the input device is configured to receive a search query from the user, and wherein the processor is configured to search for one of the first and second stored EGM waveforms based on the search query.

18. The computing device of claim 1, wherein the processor is configured to store image files in association with at least one of the first and second stored EGM waveforms in the memory device.

19. A method comprising:
wirelessly receiving a stream of first electrogram (EGM) waveforms of a patient from an implantable medical device (IMD);
receiving a stream of second EGM waveforms of the patient that are based on external measurement of changes in electrical potential associated with the patient;
storing the first and second EGM waveforms in a memory device;
displaying the first and second EGM waveforms together on a display when the first and second EGM waveforms are received;
receiving a selection command from a user;
retrieving a portion of the first stored EGM waveforms from the memory device in response to the selection command; and
displaying the retrieved portion of the first stored EGM waveforms on the display while concurrently storing the stream of the first EGM waveforms in the memory device.

20. The method of claim 19, further comprising:
wirelessly receiving marker channel data from the IMD that indicates the occurrence and timing of therapy and sensing events associated with the IMD;
storing the marker channel data in the memory device;
displaying the marker channel data on the display together with the first and second EGM waveforms when the marker channel data is received;
retrieving a portion of the marker channel data from the memory device in response to the selection command; and
displaying the retrieved portion of the marker channel data on the display while concurrently storing received marker channel data in the memory device.

21. The method of claim 19, wherein the first EGM waveforms comprise cardiac electrogram (CEGM) waveforms, and the second EGM waveforms comprise electrocardiogram (ECG) waveforms.

22. The method of claim 19, further comprising:
receiving an annotation from a user; and
displaying the annotation on a selected portion of one of the first and second EGM waveforms displayed while concurrently storing the streams of the first and second EGM waveforms in the memory device.

23. A computer-readable storage medium comprising instructions that cause a programmable processor to:
wirelessly receive a stream of first electrogram (EGM) waveforms of a patient from an implantable medical device (IMD);
receive a stream of second EGM waveforms of the patient that are based on external measurement of changes in electrical potential associated with the patient;
store the first and second EGM waveforms in a memory device;
display the first and second EGM waveforms together on a display when the first and second EGM waveforms are received;
receive a selection command from a user;
retrieve a portion of the first stored EGM waveforms from the memory device in response to the selection command; and
display the retrieved portion of the first stored EGM waveforms on the display while concurrently storing the stream of the first EGM waveforms in the memory device.

24. The computer-readable storage medium of claim 23, further comprising instructions that cause the programmable processor to:
wirelessly receive marker channel data from the IMD that indicates the occurrence and timing of therapy and sensing events associated with the IMD;
store the marker channel data in the memory device;
display the marker channel data on the display together with the first and second EGM waveforms when the marker channel data is received;
retrieve a portion of the marker channel data from the memory device in response to the selection command; and
display the retrieved portion of the marker channel data on the display while concurrently storing received marker channel data in the memory device.

25. The computer-readable storage medium of claim 23, further comprising instructions that cause the programmable processor to:
receive an annotation from a user; and
display the annotation on a selected portion of one of the first and second EGM waveforms displayed while concurrently storing the streams of the first and second EGM waveforms in the memory device.

26. A system comprising:
means for wirelessly receiving a stream of first electrogram (EGM) waveforms of a patient from an implantable medical device (IMD);
means for receiving a stream of second EGM waveforms of the patient that are based on external measurement of changes in electrical potential associated with the patient;
means for storing the first and second EGM waveforms in a memory device;
means for displaying the first and second EGM waveforms together on a display when the first and second EGM waveforms are received;
means for receiving a selection command from a user;
means for retrieving a portion of the first stored EGM waveforms from the memory device in response to the selection command; and
means for displaying the retrieved portion of the first stored EGM waveforms on the display while concurrently storing the stream of the first EGM waveforms in the memory device.

27. A system comprising:
a memory device;
an interface configured to wirelessly receive, from a medical device, a stream of waveforms that represent a physiological characteristic of a patient;
a processor configured to store the waveforms in the memory device and retrieve the stored waveforms from the memory device;
a display controller configured to display the waveforms on a display when the waveforms are received; and
an input device configured to receive a selection command from a user, wherein the processor retrieves a portion of the waveforms from the memory device in response to the selection command, and wherein the display controller displays the retrieved portion of the waveforms on the display while the processor concurrently stores the stream of the waveforms in the memory device.

28. The system of claim 27, wherein the medical device comprises an implantable medical device.

29. The system of claim 27, wherein the waveforms include sensor waveforms from a sensor that senses physiological characteristics of the patient.

30. The system of claim 27, wherein the waveforms include electrogram waveforms.

31. The system of claim 30, wherein the waveforms include cardiac electrogram waveforms.
